(19) United States
(12) Patent Application Publication
(10) Pub. No.: US 2026/0069338 A1
(43) Pub. Date: Mar. 12, 2026

(54) SINGLE USE SURGICAL MALLET

(71) Applicant: Mainstream Medical Devices, LLC, Austin, TX (US)

(72) Inventors: Aaron Bailey, Austin, TX (US); Michael Seikel, Austin, TX (US)

(21) Appl. No.: 19/319,315

(22) Filed: Sep. 4, 2025

Related U.S. Application Data
(60) Provisional application No. 63/693,810, filed on Sep. 12, 2024.

(51) Int. Cl. A61B 17/92; A61B 17/00

(52) U.S. Cl. CPC A61B 17/92; A61B 2017/00464; A61B 2017/00469; A61B 2017/00526; A61B 2017/922

(57) ABSTRACT

A single-use surgical mallet of modular construction having a handle and a hammer head insert, which engages the handle. In an embodiment, the handle may be an injection molded unit. Injection molding the handle allows producing the handle in a cost-efficient manner. The handle includes a receiving recess or socket for receiving the hammer head insert. Hammer head inserts may be made in a variety of different weights and/or materials while having a substantially similar or identical exterior configuration. In this regard, differently weighted hammer head inserts are configured to engage a common handle allowing the production of differently weighted mallets using the same handle.

### CROSS REFERENCE

This application claims the benefit of the filing date of U.S. Provisional Patent No. 63/693,810 having a filing date of Sep. 12, 2024, the entire contents of which is incorporated herein by reference.

### FIELD

The present disclosure is directed to single-use medical devices and implements (hereafter “devices”). More specifically, the present disclosure is directed to single-use surgical devices.

### BACKGROUND

Historically, medical devices, such as those utilized in surgery (e.g., mallets, scalpels, forceps, spreaders, scrapers, etc.) were reusable. That is, the devices were intended to be re-sterilized/decontaminated and reused after a medical procedure. Such re-sterilization has advanced over time from simply immersing the devices or tools in alcohol to the current practice of autoclave sterilization. In the latter regard, medical devices are inserted into an autoclave chamber where they are subjected to elevated temperatures, pressures and, commonly, steam.

While sterilization and reuse of medical devices is an industry accepted standard, there are instances where reuse of medical devices is not desirable. For instance, many medical procedures are performed in out-patient clinics that may not have the ability to effectively sterilize some medical devices. Accordingly, there has been an ongoing shift in the medical field to single-use medical devices. Such devices are designed to be utilized once in a hospital or clinic and then disposed. In this regard, single-use devices are often delivered in sterilized packages, which are opened shortly before or during a medical procedure. In such an arrangement, no sterilization is required before use. Such single-use devices eliminate re-sterilization problems sometimes encountered by reusable medical devices.

### BRIEF SUMMARY OF THE DISCLOSURE

The present inventor(s) have recognized that, while many hospitals and clinics desire single-use medical devices, some medical devices do not lend themselves to single-use configurations due to the cost of making each device. For instance, surgical mallets are typically available in a variety of weights, and have historically required the separate manufacture of multiple mallets to accommodate each weight. This individualized production approach significantly increases the per-unit cost and has made single-use mallets prohibitively expensive for many end users.

Presented herein is a single-use surgical mallet that is of modular construction having a handle and a hammer head insert, which engages the handle. In an embodiment, the handle may be an injection molded unit. Injection molding the handle allows producing the handle assembly in a cost-efficient manner. The handle includes an elongated grip and a receiving recess or socket for receiving the hammer head insert. Hammer head inserts may be made in a variety of different weights and/or materials while having a substantially similar or identical exterior configuration. In this regard, differently weighted hammer head inserts are configured to engage a common handle allowing the production of differently weighted mallets using the same handle. In an embodiment, hammer head inserts having different weights may be formed in a cost-efficient manner in various processes. For instance, such hammer head inserts may be milled, machined, metal-injection molded and/or cast, among others. This allows for producing a set of differently weighted mallets in an economical process that allows the mallets to be used in single-use applications.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIGS. 1A and 1B illustrate side and end plane views, respectively of a single-use surgical mallet, in an embodiment.

FIG. 2 illustrates an exploded perspective view of the mallet of FIGS. 1A and 1B.

FIG. 3 illustrates a cross-sectional view of the mallet of FIG. 1B.

FIGS. 4A-4D illustrate a hammer head insert, and three cross-sectional views of the hammer head insert having different weights.

FIGS. 5A and 5B illustrate perspective and side views, respectively, of another embodiment of a single-use surgical mallet.

FIGS. 6A and 6B illustrate perspective and side exploded views, respectively, of the single-use surgical mallet of FIGS. 5A and 5B.

FIG. 7 illustrates use of the mallet of FIGS. 5A and 5B used as a slap hammer.

FIG. 8 illustrates another embodiment of a hammer head insert.

### DETAILED DESCRIPTION

Reference will now be made to the accompanying drawings, which will at least assist in illustrating the various pertinent features of the presented inventions. The following description is presented for purposes of illustration and description and is not intended to limit the inventions to the forms disclosed herein. Consequently, variations and modifications commensurate with the following teachings, and skill and knowledge of the relevant art, are within the scope of the presented inventions. The embodiments described herein are further intended to explain the best modes known of practicing the inventions and to enable others skilled in the art to utilize the inventions in such, or other embodiments and with various modifications required by the particular application(s) or use(s) of the presented inventions.

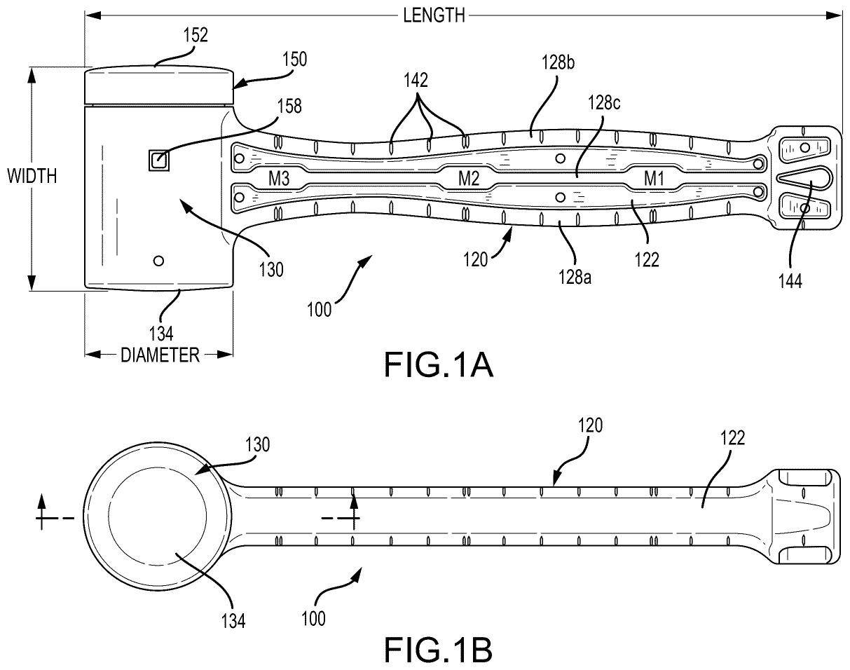

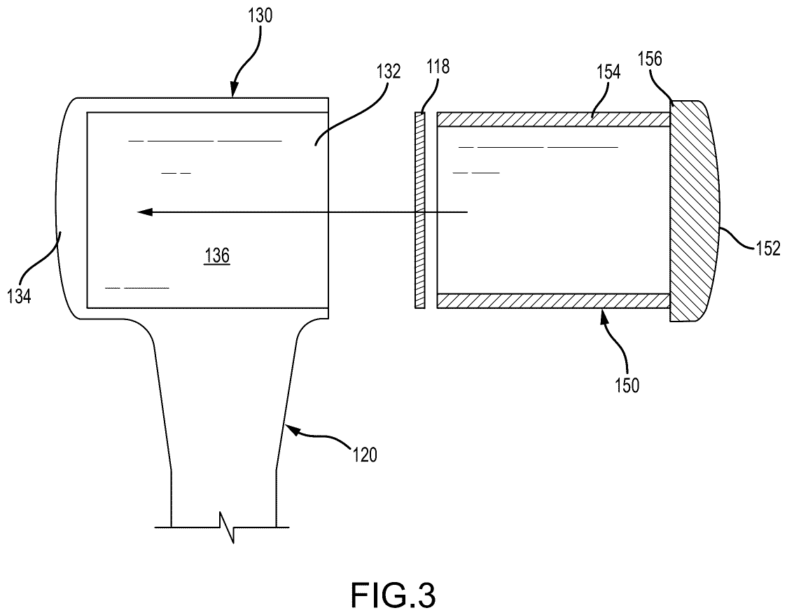

FIGS. 1A, 1B, 2 and 3 illustrate one embodiment of a single use surgical mallet 100. As shown, the surgical mallet 100 has a modular construction with a handle 120 and a hammer head insert 150 (hereafter “head insert”). As shown, the handle 120 includes an elongated grip 122 having a free end 124 and a second end 126 fixedly connected to a socket 130 that is configured to receive the head insert 150. As best shown in the cross-sectional view of FIG. 3, the socket 130 is a generally hollow element having an open end 132 and a closed-end 134. A sidewall of the socket 130 surrounds a hollow interior 136. As illustrated, the socket 130 has a generally cylindrical hollow interior 136. However, it will be appreciated that in other embodiments the interior of the socket 130 may have other shapes (e.g., square hexagonal etc.). As shown, the hollow interior 136 of the socket 130 is sized to receive therein a portion of the head insert 150. More specifically, a sleeve 154 of the head insert 150 slides into the interior of the socket 130.

In the illustrated embodiment, the handle 120 is an integrally formed injection molded unit. Typically, the handle 120 is formed of a polymeric material. Of note, the elongated grip 122 includes two outer ribs 128a, 128b and a center rib 128c. These ribs 128 are integrally connected along their midline as best illustrated in FIG. 1A. The thickness of the ribs 128 as well as the sidewall of the socket 130 may be formed to have substantially equal thicknesses that facilitate the injection molding process.

In the illustrated embodiment, the handle 120 includes a number of optional features. As best illustrated in FIG. 1A, the elongated grip 122 may include measurement markings M1, M2, M3, etc. along its length. Such measurement markings M1, M2 M3, etc. may indicate a length dimension from the free end 124 of the elongated handle 122. In the illustrated embodiment, the measurement markings M1, M2 and M3 may provide equal 50 mm measurements from the free end 124 of the grip 122. Other dimensions and units of measure are possible. These measurement markings may be utilized by a clinician during a surgical procedure. In addition, the grip 122 may have spaced protruding serrations 142 formed on the outer ribs 128a, 128b of the grip 122. The serrations 142 provide an additional grasp when a clinician grasps the elongated grip 122. Finally, in the illustrated embodiment, free end 124 of the elongated grip 122 includes a pin pulling aperture 144. The pin pulling aperture is generally teardrop shaped with a narrower end and a wider end. This pin pulling aperture 144 allows the clinician to place a pin through the wide end of the aperture and utilize the narrow end to pull on the pin.

The head insert 150 includes a hammer head 152 and a sleeve 154 fixedly attached thereto. The sleeve 154 has an outer periphery that is sized for receipt (e.g., conformal receipt) within the hollow interior 136 of the socket 130. In embodiment, the outer periphery of the sleeve 154 has the same shape as the interior periphery of the socket 130. This allows the sleeve 154 to be slidably received within the hollow interior 136 of the socket 130. In the illustrated embodiment, an outside dimension (e.g., diameter) of the sleeve 154 is less than the outside dimension of the solid hammer head 152 such that a lip 156 is formed therebetween. The lip 156 abuts against the end of the sidewall about the open end 132 of the socket 130 when the head insert 150 is inserted within the socket 130.

In the illustrated embodiment, at least one protrusion 158 is formed on an exterior surface of the sleeve 154. An additional protrusion may be formed on opposing surfaces of the head insert 150 (not shown). The protrusion(s) 158 extends above the peripheral surface of the sleeve 154 and is/are configured for receipt within an aperture(s) 138 formed in or through a sidewall of the socket 130. In this regard, when the head insert 150 is disposed into the socket 130, the protrusion(s) slightly expands the sidewall of the socket 130 until received within the aperture(s) 138. Once seated within the aperture(s) 138, the protrusion(s) 158 fixes the insert 150 within the socket 130 of the handle 120. That is, the protrusion(s) 158 and aperture(s) 138 provides a snap for connection between the handle 120 and the head insert 150. In addition, or alternatively, one or more of various types of screws, in this instance set screws 148, may extend through the sidewall of the socket 130 and engage the sleeve 154 of the head insert 150. The set screws may be received within threaded apertures or indentions formed on or in the sleeve 154.

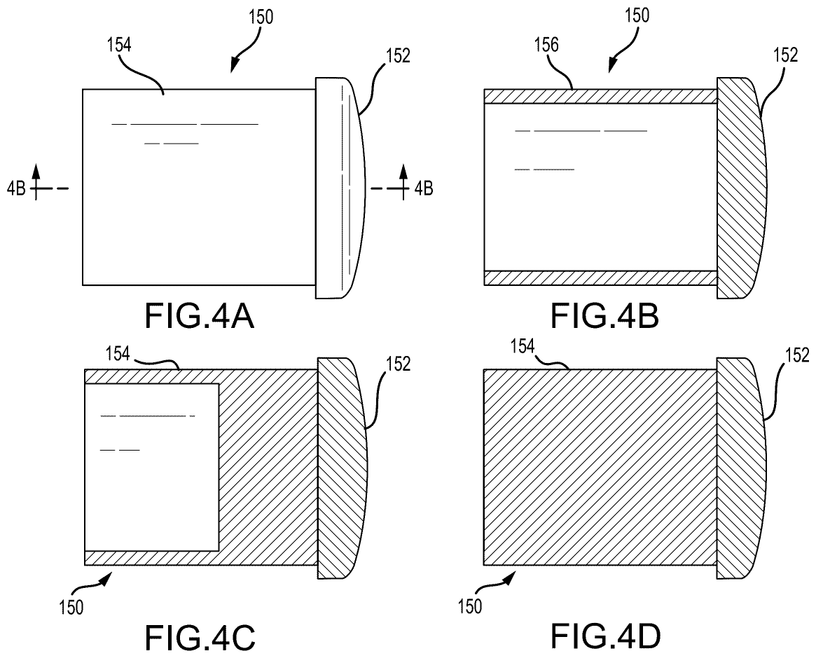

Hammer head inserts 150 may be made in a variety of different weights while having a substantially similar or identical exterior configuration. In this regard, differently weighted head inserts 150 are configured to engage a common handle 120 allowing the production of differently weighted mallets 100 using the same handle 120. In an embodiment, hammer head inserts 150 having different weights may be formed in a cost-efficient manner. For instance, such hammer head inserts may be milled, machined, metal-injection molded and/or cast, among other methodologies. FIGS. 4A-4D illustrate three embodiments of head inserts 150 having three different weights. More specifically, FIGS. 4B, 4C and 4D are alternate cross-sectional views of the head insert 150 of FIG. 4A. As shown, the head insert 150 of FIG. 4B includes a mostly hollow sleeve 154. In contrast the sleeve 154 of the insert 150 of FIG. 4C is half filled/half solid. Finally, the sleeve 154 of the insert 150 of FIG. 4D is solid. By way of example only, the insert of FIG. 4A may form an 8-ounce mallet, the insert a FIG. 4B may form a 12-ounce mallet and the insert a FIG. 4D may form a 16-ounce mallet. The insert 150 may be hollowed from different orientations other than the one shown. Alternatively, different materials may be used to form head inserts that have different weights. For example, the head insert 150 of FIG. 4D made of steel may form a 16-ounce insert while an identical head insert made of aluminum may form an 8-ounce insert. Other weights and/or materials are possible. As will be appreciated while having different weights, each of the inserts includes a common exterior shape of at least the outer periphery of the sleeve allowing each of the inserts to be utilized with a common handle.

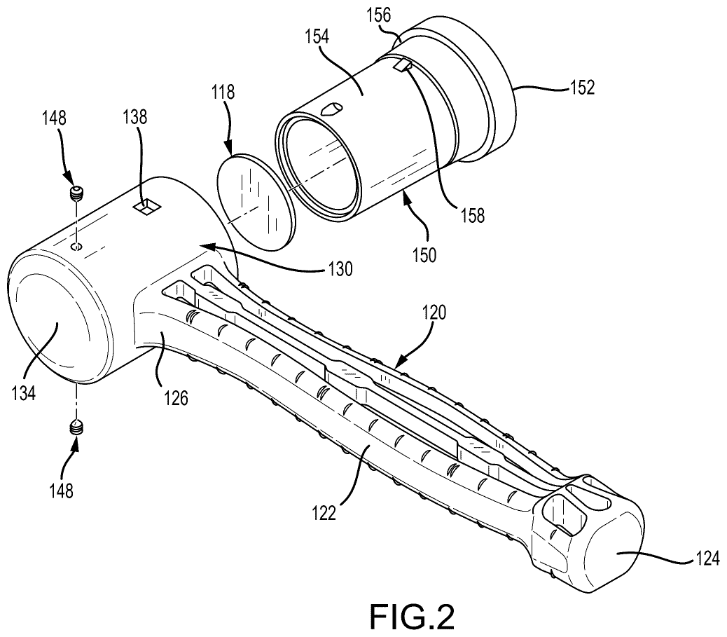

Referring to FIGS. 2 and 3, an additional element that may be utilized with the mallet 100 is illustrated. Specifically, when utilizing a head insert 150 having a partially hollow interior (e.g., FIGS. 4B, 4C), it may be desirable provide an end cap 118 that extends over the open end of the sleeve 154 of the insert 150. More specifically, when the insert 150 is inserted within the handle 120 the solid hammer head 152 of the insert forms one face of the mallet 100 while the closed end 134 of the socket 130 forms a second face of the mallet 100. The second face of the mallet 100 in this embodiment is formed of a plastic or polymeric material and would deflect without sufficient backing. When used, the end cap 118 abuts the open end of the sleeve 154. Additionally, the end cap 118, which may be a metallic element, provides a solid backing for the second face of the mallet 100. As will be appreciated, in such an embodiment, the mallet includes a metallic face and a softer polymeric face. In use, a clinician may prefer one face or the other depending on their needs. For instance, the polymeric face may provide a reduced level of sound in use compared to the metallic face, as well as provide protection for the device being struck by the mallet.

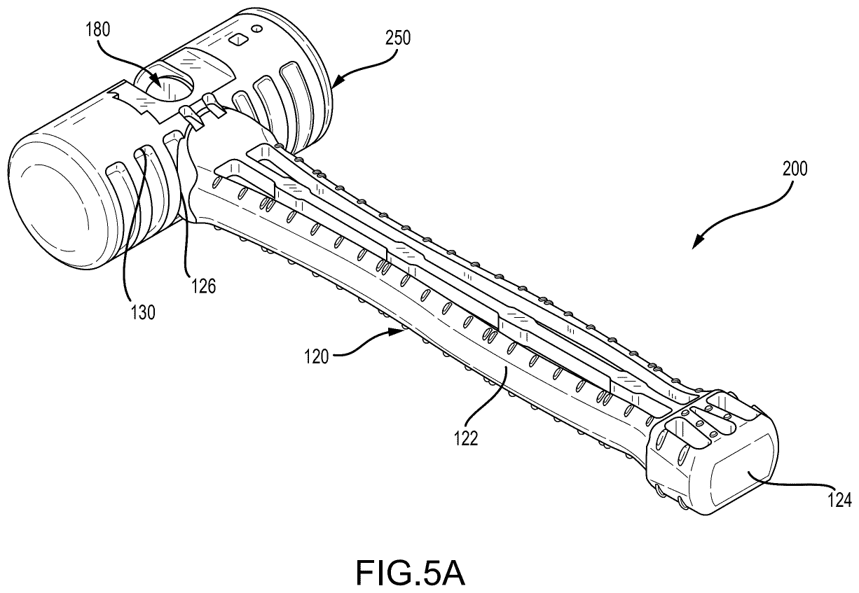

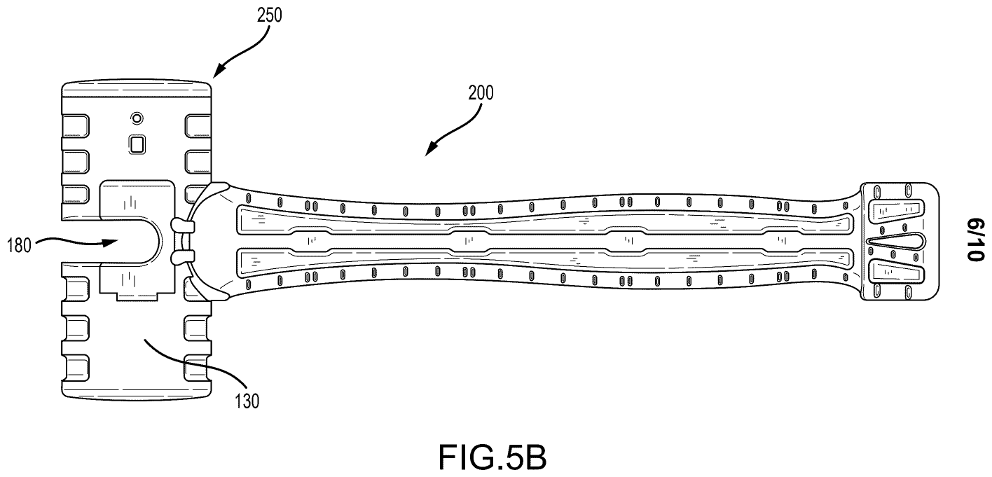

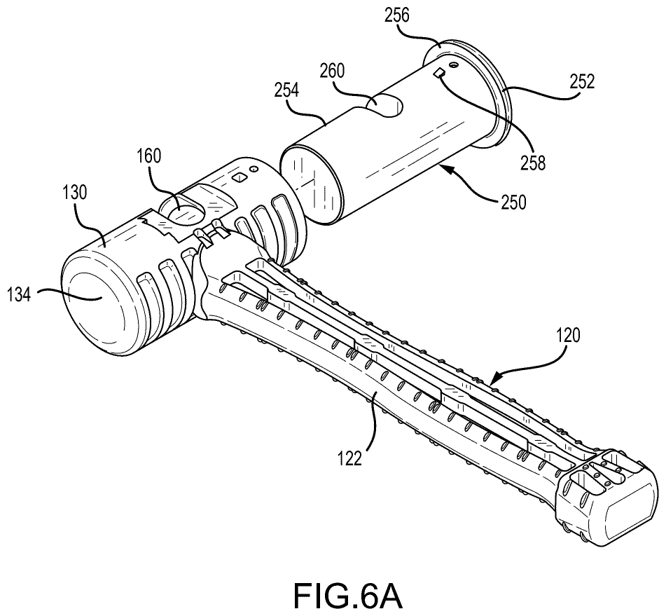

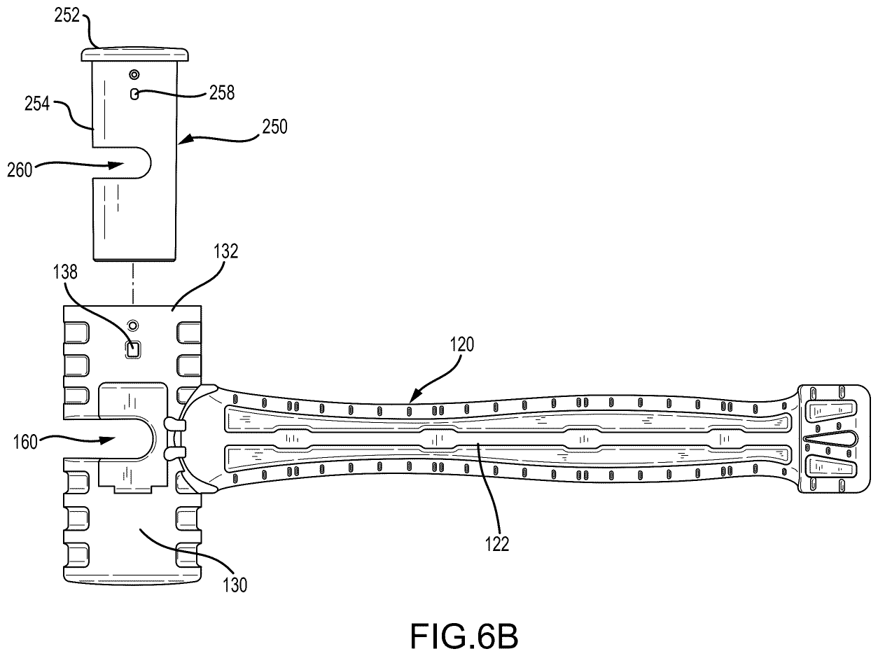

FIGS. 5A-6B variously illustrate another embodiment of a single use surgical mallet 200. The illustrated mallet 200 includes many common features with the mallet 100 illustrated in FIGS. 1A-3 and these common features have common reference numbers. Generally, the mallet 200 is of a modular construction having a handle 120 and a head insert 250. As shown, the handle 120 includes an elongated grip 122 having a free end 124 and a second end 126 fixedly connected to a socket 130, which is configured to receive the head insert 250. The socket 130 is a generally hollow element having an open end 132, a closed-end 134 and a hollow interior (not shown). The hollow interior of the socket 130 is sized to receive a sleeve portion 254 of the head insert 250. The head insert also includes a solid hammer head 252 attached to one end of the sleeve 254 fixedly attached thereto. The sleeve 154 has an outer periphery that is sized for receipt (e.g., conformal receipt) within the hollow interior of the socket 130. As the illustrated embodiment, an outside dimension (e.g., diameter) of the sleeve 254 is less than the outside dimension of the solid hammer head 252 such that a lip 256 is formed therebetween. The lip 256 is proximate to the end of the sidewall about the open end 132 of the socket 130, when the head insert 250 is inserted within the socket 130. Typically, the solid end or end cap (if utilized) of the insert 250 comes into full contact and mates with the closed end 134 of the socket 130, which forms the polymeric strike face, when the head insert 250 is inserted within the socket 130. One or more protrusion 258 may be formed on an exterior surface of the sleeve 254, which are configured for receipt within one or more mating aperture 138 formed in or through a sidewall of the socket 130. As with the embodiment discussed above, different head inserts 250 may have different weights while be compatible with a common handle.

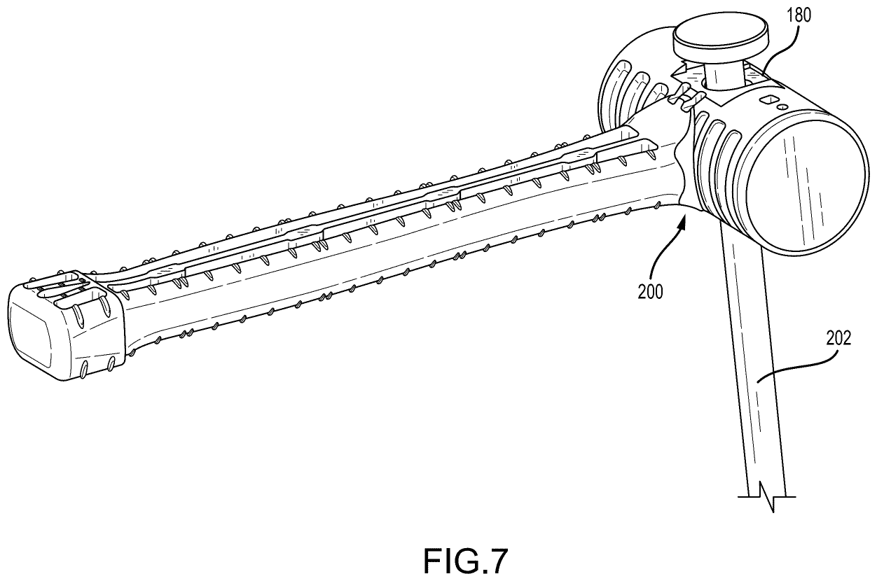

The mallet 200 illustrated in FIGS. 5A-6B is a slotted mallet having a slap slot 180. The slap slot 180 is collectively defined by a slap slot 160 formed in the socket 130 and a slap slot 260 formed in the sleeve 254 of the head insert 250. When the sleeve 254 of the head insert 250 is inserted into the socket 130, the slap slots 160, 260 align to form the slap slot 180. The slap slot 180 allows using the mallet 200 like a slap hammer. That is, the mallet 200 may slide along a rod 202 disposed within the slap slot 180 and impact an abutment on either end of the rod 202. See FIG. 7.

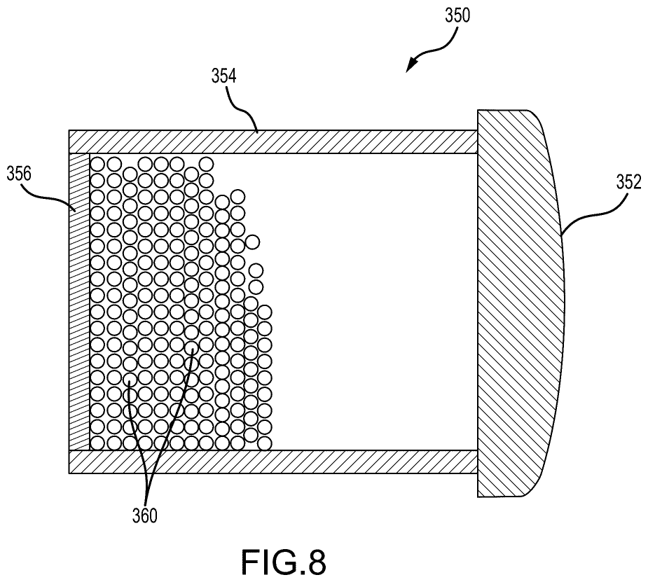

FIG. 8 illustrates a cross-sectional side view of another embodiment of a head insert 350, which may be used with a handle. In this embodiment, the head insert includes a hollow sleeve 354 attached to a hammer head 352. A cap 356 encloses the interior of the hollow sleeve 354. Loose material 360 is disposed within an interior of the hollow sleeve 354. For instance, beads or shot may be disposed in the hollow sleeve 354, among other materials. Typically, such loose material only partially fills the hollow interior of the sleeve. In use, the loose material makes a mallet using the head insert 350 a dead blow mallet. Such dead blow mallets are sometimes used to deliver blows without damaging a target surface. Dead blow mallets with moving parts such as the shot in the hollow sleeve 354, are known to greatly reduce shock and reduce rebound when striking objects with the mallet. This feature may be greatly appreciated in more delicate surgical applications.

All directional references (e.g., distal, proximal, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the any aspect of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A surgical mallet, comprising:
a handle having:
an elongated grip;
a socket fixedly attached to one end of the elongated grip, the socket having an at least partially hollow interior;
a head insert having:
a hammer head; and
a sleeve, wherein the sleeve is configured for slidable receipt within at least a portion of the interior of the socket.

2. The surgical mallet of claim 1, wherein the socket includes an open end and a closed end and a sidewall, wherein the sidewall defines an interior periphery of the socket.

3. The surgical mallet of claim 2, wherein an outer periphery of the sleeve of the head insert has a common shape with the interior periphery of the socket.

4. The surgical mallet of claim 1, wherein an outer dimension of the sleeve is less than an outer dimension of the hammer head, wherein a lip is formed between the sleeve and the hammer head.

5. The surgical mallet of claim 4, wherein the lip of the head insert abuts an end of the socket when the sleeve is inserted into the socket.

6. The surgical mallet of claim 1, wherein the handle is an integrally formed injection molded unit.

7. The surgical mallet of claim 6, wherein the handle is polymeric.

8. The surgical mallet of claim 7, wherein the handle is formed from machining, from 3D printing, from molding, and/or a combination thereof.

9. The surgical mallet of claim 1, wherein the head insert is metallic.

10. The surgical mallet of claim 9, wherein the head insert is formed from a casting, from machining, from 3D printing, from molding, and/or a combination thereof.

11. The surgical mallet of claim 1, wherein the sleeve comprises at least one protrusion extending above its outer surface, wherein the protraction is configured for receipt within a recess or aperture formed in an interior surface of the socket.

12. The surgical mallet of claim 1, wherein the sleeve has at least partially hollow interior.

13. The surgical mallet of claim 12, wherein loose material at least partially fills a hollow interior of the sleeve.

14. The surgical mallet of claim 12, further comprising an end cap covering an opening to the at least partially hollow interior of the sleeve.

15. The surgical mallet of claim 14, wherein the end cap is disposed between a free end of the sleeve and a bottom surface of the socket.

16. The surgical mallet of claim 1, wherein a first face of the mallet is a metallic face and a second face of the mallet is a polymeric face.

17. The surgical mallet of claim 1, further comprising a slap slot.

18. The surgical mallet of claim 17, wherein the socket includes a first slap slot and the head insert includes a second slap slot, wherein the first and second slap slots are at least partially aligned when the sleeve is received within the socket.

19. The surgical mallet of claim 1, further comprising:
a plurality of head inserts, each head insert having a different weight while being configured for receipt within the socket.

20. A method of forming a surgical mallet according to claim 18, further comprising:
selecting a head insert of a desired weigh; and
inserting the sleeve of the selected head insert into the socket of the handle.
